(19) United States
(12) Patent Application Publication
(10) Pub. No.: US 2022/0249330 A1
(43) Pub. Date: Aug. 11, 2022

(54) Bluetooth Pacifier

(71) Applicants: Ted Romero, Morrison, CO (US); William Romero, Lakewood, CO (US)

(72) Inventors: Ted Romero, Morrison, CO (US); William Romero, Lakewood, CO (US)

(21) Appl. No.: 17/385,162

(22) Filed: Jul. 26, 2021

Related U.S. Application Data
(60) Provisional application No. 63/148,176, filed on Feb. 11, 2021.

(51) Int. Cl. A61J 17/00; G16H 40/63

(52) U.S. Cl. CPC A61J 17/001; G16H 40/63; A61J 17/103

(57) ABSTRACT

The present invention relates to a Bluetooth pacifier for infants, toddlers, and children. More specifically, the Bluetooth pacifier includes a nipple, a collar and a handle. The Bluetooth pacifier includes an integrated Bluetooth microchip and a micro-USB charging port in the collar. The Bluetooth microchip periodically transmits Bluetooth Low Energy signals (BLE) to a paired electronic device. A software application installed in the electronic device processes the received BLE signals and associated RSSI values of the BLE signals for location tracking of the pacifier. When the pacifier and the electronic device are not in BLE range, the software application stores the last recorded (i.e. past-time) location of the pacifier. The micro-USB charger can be used for attaching a USB charger.

### CROSS-REFERENCE TO RELATED APPLICATION

The present application claims priority to, and the benefit of, U.S. Provisional Application No. 63/148,176 which was filed on Feb. 11, 2021 and is incorporated herein by reference in its entirety.

### FIELD OF THE INVENTION

The present invention relates generally to the field of baby pacifiers. More specifically, the present invention relates to a uniquely-designed pacifier equipped with a Bluetooth microchip and a micro-USB charging port, to allow a user to track and locate a lost or dropped pacifier. The Bluetooth pacifier allows the users to connect the pacifier to a companion software application to identify the exact location of the lost pacifier. Therefore, the invention alleviates the frustration that children encounter upon misplacing or losing their pacifier. The invention enables the users to quickly locate the pacifiers and prevents the users from expending unnecessary time in searching for the lost pacifiers, thereby saving time and frustration for the users. Accordingly, the present disclosure makes specific reference thereto. Nonetheless, it is to be appreciated that aspects of the present invention are also equally applicable to other like applications, devices, and methods of manufacture.

### BACKGROUND OF THE INVENTION

By way of background, a pacifier is a rubber, plastic or silicone nipple that is given to an infant or other young child for oral stimulation. Pacifiers are used for calming down crying infants when they are hungry, too full to eat, uncomfortable or in need of the comfort that sucking and oral stimulation provides. Additionally, pacifiers are used to protect an infant from sudden infant death syndrome (SIDS) and suffocation during sleep. Further, pacifiers can be used to distract babies and infants when they are unwell, going for health check-ups, travelling or in other similar situations.

The standard pacifier comprises a nipple, a guard and a ring attached to the guard. The nipple is inserted into an infant's mouth to calm the baby down and to ensure the infant stays preoccupied and busy, while nearby adults can easily work on other tasks. Babies, toddlers and other young children who use pacifiers may accidently drop their pacifier on the floor, which may bounce and roll underneath furniture or get kicked by the child and inevitably end up hidden, lost or misplaced. Occupied individuals may not be able to constantly monitor the babies and may not be aware of where the pacifier might have been lost, misplaced or dropped, while they were concentrating on other tasks. To locate a lost pacifier, individuals may expend considerable time looking for it and may have to leave their current tasks. Individuals may easily get frustrated while looking for the misplaced pacifiers. Additionally, infants can quickly get upset if they cannot find their pacifiers immediately, and may start screaming or crying, which causes additional frustration for nearby adults.

Therefore, there exists a long felt need in the art for a smart pacifier device that can be easily tracked and located when the pacifier is accidently dropped, misplaced, or lost by the infants. There is also a long felt need in the art for a smart pacifier that ensures adults can quickly and easily locate a lost or misplaced pacifier. Additionally, there is a long felt need in the art for a pacifier that enables the users to identify the exact location of the lost or misplaced pacifiers without expending a great deal of time looking for the lost or misplaced pacifiers, thereby saving time for the adults. Moreover, there is a long felt need in the art for a pacifier device that ensures babies and infants get the lost or misplaced pacifier returned in a short period of time. Further, there is a long felt need in the art for a pacifier device that allows adults to work on other tasks without worrying about the pacifiers becoming lost or misplaced. Finally, there is a long felt need in the art for a smart pacifier device that prevents children from becoming upset due to lost or misplaced pacifiers that cannot be found in a timely manner.

The subject matter disclosed and claimed herein, in one embodiment thereof, comprises a Bluetooth-capable pacifier. The pacifier comprises a nipple, a collar and a handle. More specifically, the pacifier includes an integrated Bluetooth microchip and a micro-USB charging port in the collar. The Bluetooth microchip periodically transmits Bluetooth Low Energy signals (BLE) to a paired electronic device, and a software application installed in the electronic device processes the received BLE signals and associated ‘received signal strength indicator’ (RSSI) values of the BLE signals for location tracking of the pacifier. RSSI can provide a measurement of how fell the paired electronic device can hear a signal from the BLE signal. It is a value that is useful for determining if you have enough signal to get a good wireless connection. When the pacifier and the electronic device are not in BLE range, the software application stores the last recorded location of the pacifier. The micro-USB charger can be used for attaching a USB charger. The Bluetooth pacifier enables parents to easily locate the lost or misplaced pacifier using the real-time or last recorded location of the pacifier.

In this manner, the novel Bluetooth pacifier of the present invention accomplishes all of the forgoing objectives, and provides a relatively easy, convenient and effective solution to track and locate pacifiers inevitably lost, misplaced or dropped by infants and toddlers. The Bluetooth pacifier of the present invention is also user friendly, as it does not require the user to put much effort or labor into locating lost pacifiers, and saves the time of the users. The Bluetooth pacifier allows the users to monitor a pacifier through a companion software application while working on other tasks.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a Bluetooth-capable pacifier. The pacifier comprises a nipple, a collar and a handle. More specifically, the pacifier includes an integrated Bluetooth microchip and a micro-USB charging port in the collar. The Bluetooth microchip periodically transmits Bluetooth Low Energy signals (BLE) to a paired electronic device, and a software application installed in the electronic device processes the received BLE signals and associated Received Signal Strength Indicator (RSSI) values of the BLE signals for location tracking of the pacifier. RSSI can provide a measurement of how well the paired electronic device can hear a signal from the BLE signal. It is a value that is useful for determining if you have enough signal to get a good wireless connection. When the pacifier and the electronic device are not in BLE range, the software application stores the last recorded location of the pacifier. The micro-USB charger can be used for attaching a USB charger. The Bluetooth pacifier enables parents to easily locate the lost or misplaced pacifier using the real-time or last recorded location of the pacifier.

In a further embodiment of the present invention, a pacifier that provides soothing to a baby, infant, or child is disclosed. The pacifier comprises a nipple, a hard base or collar and a pivotable handle. The collar includes an integrated Bluetooth microchip and a micro-USB charging port. The Bluetooth microchip is configured to periodically transmit Bluetooth Low Energy signals (BLE) to a paired electronic device. The BLE signals are processed by a software application installed in the electronic device and provide to the user a map that shows the location of the pacifier on the display of the electronic device. The location identifier improves navigation and reduces time spent on searching for a lost, misplaced or dropped pacifier.

In a further embodiment of the present invention, the Bluetooth pacifier can be utilized by children and toddlers of various ages. The Bluetooth pacifier can be sterilized as needed, and the nipple can be replaced as per the preferences of the children. The Bluetooth pacifier can be tracked and located easily using Bluetooth Low Energy (BLE) signals transmitted from an integrated Bluetooth microchip.

In yet a further embodiment, a method of locating a pacifier in an indoor environment is described. The method includes the initial step of pairing an electronic device having a computer-implemented software application with the pacifier using Bluetooth signals. Then, periodic Bluetooth low energy signals are transmitted from an integrated Bluetooth chip in the pacifier to the electronic device. Next, received Bluetooth low energy signals are processed by the software application to determine a location of the pacifier, and the location is then shown on a map displayed on the display screen of the electronic device. The Bluetooth pacifier enables parents to easily locate a lost, misplaced, or dropped pacifier, eliminating the need to expend considerable time searching for the pacifier. The pacifier further offers a way to prevent children from becoming upset due to misplacement of their pacifier and the inability to find their pacifier.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the Bluetooth baby pacifier of the present invention in accordance with the disclosed architecture;

FIG. 2 illustrates a schematic diagram showing connection of the Bluetooth baby pacifier of the present invention with a companion smartphone application installed on an electronic device in accordance with the disclosed architecture;

FIG. 3 illustrates a close-up perspective view of one potential embodiment of the Bluetooth microchip and micro-USB charging port of the Bluetooth pacifier of the present invention in accordance with the disclosed architecture;

FIG. 4 illustrates a flow diagram showing tracking of the location of the Bluetooth baby pacifier of the present invention using Bluetooth signals in accordance with the disclosed architecture;

FIG. 5 illustrates a perspective view showing the last recorded location of a connected Bluetooth pacifier on display of an electronic device using smartphone application in accordance with the disclosed architecture; and

FIG. 6 illustrates a perspective view of the activated integrated Bluetooth chip of the Bluetooth pacifier of the present invention to transmit real-time location in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a smart pacifier device that can be easily tracked and located when the pacifier is accidently dropped, misplaced, or lost by infants. There is also a long felt need in the art for a smart pacifier that ensures adults can quickly and easily locate a lost or misplaced pacifier. Additionally, there is a long felt need in the art for a pacifier that enables the users to identify the exact location of the lost or misplaced pacifiers. without expending a great deal of time looking for the lost or misplaced pacifiers, and therefore saves time for the adults. Moreover, there is a long felt need in the art for a pacifier device that ensures babies and infants get the lost or misplaced pacifier returned in a short period of time. Further, there is a long felt need in the art for a pacifier device that allows adults to work on other tasks, without worrying about the pacifiers becoming lost or misplaced. Finally, there is a long felt need in the art for a smart pacifier device that prevents children from becoming upset due to lost or misplaced pacifiers that cannot be found in a timely manner.

The present invention, in one exemplary embodiment, comprises a Bluetooth capable pacifier. The pacifier comprises a nipple, a collar and a handle. More specifically, the pacifier includes an integrated Bluetooth microchip and a micro-USB charging port in the collar. The Bluetooth microchip periodically transmits Bluetooth Low Energy signals (BLE) to a paired electronic device, and a software application installed in the electronic device processes the received BLE signals and associated ‘received signal strength indicator’ (RSSI) values of the BLE signals for location tracking of the pacifier. RSSI can provide a measurement of how well the paired electronic device can hear a signal from the BLE signal it is a value that is useful for determining if you have enough signal to get a good wireless connection. When the pacifier and the electronic device are not in BLE range, the software application stores the last recorded or past-time location of the pacifier. In one illustrative example, the BLE range is generally about 30 meters. The micro-USB charger can be used for attaching a USB charger. The Bluetooth pacifier enables parents to easily locate the lost or misplaced pacifier using the real-time or last recorded location of the pacifier.

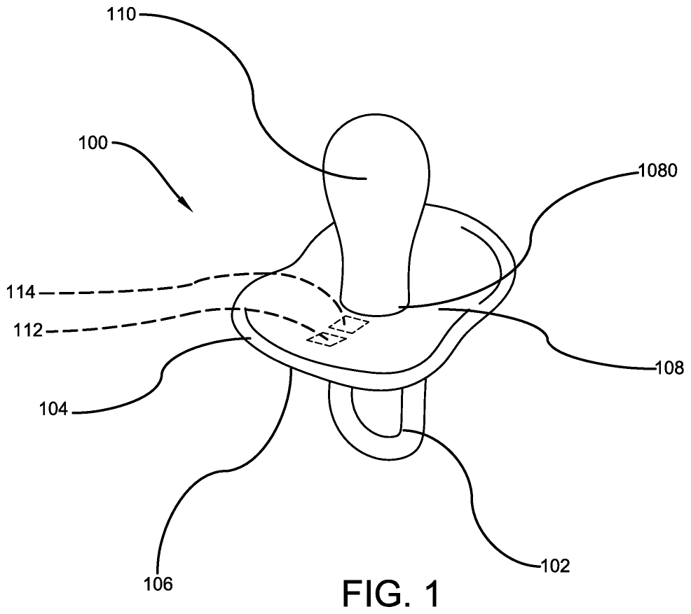

Referring initially to the drawings, FIG. 1 illustrates a perspective view of the Bluetooth baby pacifier of the present invention in accordance with the disclosed architecture. The Bluetooth baby pacifier 100 of the present invention is an improved pacifier and is configured and designed to provide the real-time location of the pacifier 100 to a pre-configured software application installed in an electronic device. The pacifier 100 can be used by infants, toddlers, or babies or other young children as a substitute for suckling in between feedings. The pacifier 100 includes a silicon or rubber nipple 110 connected to a base or a collar 104 with an integrated chin guard. The base/collar 104 can be made of plastic, rubber or any other such material. The pacifier 100 includes a handle/grip 102 which can be attached to a rear portion 106 of the base 104. The handle 102 is used for convenient carrying of the pacifier 100. The handle 102 can be magnetic to attach to a ferrous or metal surface for easy, secure and accessible storage of the pacifier 100. The handle 102 is movable and is hinged to the rear surface 106 of the collar 104 using a hinged loop 1020. The collar 104 can be of any shape such as a heart shape.

The nipple 110 is attached to the front portion 108 of the base 104 in an aperture 1080 configured and shaped to receive an open end of the nipple 110. The nipple 110 is selectively removable and replaceable with a new nipple. Specifically, the nipple 108 can be made of a transparent or translucent, non-toxic, hypo-allergenic material that can be safely introduced into an infant's mouth. The nipple 108 can have any known and/or convenient shape.

The Bluetooth baby pacifier 100 of the present invention includes a Bluetooth microchip 112 integrated into the base 104. The Bluetooth microchip 112 allows the pacifier 100 to be connected to a smartphone or a companion software application installed in one or more electronic devices. The Bluetooth microchip 112 allow one or more users, or parents, to connect a smartphone application installed in their devices to the pacifier 100 for tracking the location of the pacifier 100 through Bluetooth Low Energy (BLE) signals. The BLE enables the approximate location of the pacifier 100 to be tracked within an enclosed space such as a room or a home and allows parents, caretakers, or others to easily locate the lost, misplaced, or dropped pacifier 100, thus eliminating the need to expend considerable time searching for the pacifier 100.

It should be appreciated that a user, a parent, or any other connected device can receive the real-time location of the pacifier 100 and can track the current location of the pacifier 100 in case the pacifier is inevitably dropped or lost. The pacifier 100 includes a micro-USB charging port 114, which allows the charging of the Bluetooth microchip 112. The micro-USB charging port 114 and the Bluetooth microchip 112 are integrated in the base 104 such that they do not reduce the efficacy of the pacifier 100, and do not cause inconvenience to an infant, toddler or child using the pacifier 100.

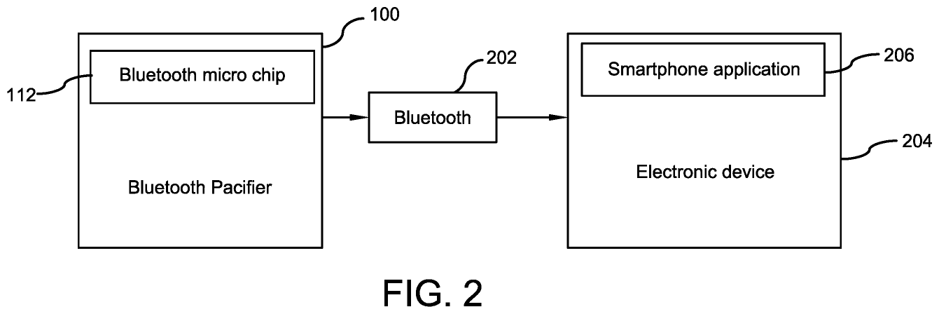

FIG. 2 illustrates a schematic diagram showing connection of the Bluetooth baby pacifier of the present invention with a companion smartphone application installed in an electronic device in accordance with the disclosed architecture. As shown, the Bluetooth pacifier 100 can connect to an electronic device 204 such as a smartphone, PDA or any other portable device using a Bluetooth Low Energy wireless communication medium 202. The Bluetooth baby pacifier 100 includes a Bluetooth microchip 112 which transmits periodic Bluetooth Low Energy signals 202 to connect and transmit location information to a smartphone application 206 installed on any electronic device 204 such as a smartphone or others. The real-time location of the Bluetooth baby pacifier 100 is identified using the received Bluetooth Low Energy signals 202. One or more users may activate the smartphone application 206 on their respective electronic devices to track and locate the pacifier 100 lost or dropped by any infant.

It should be noted that the pacifier 100 of the present invention uses Bluetooth Low Energy signals transmitted from the Bluetooth microchip 112. Bluetooth Low Energy offers low-cost, energy-efficient and easy to deploy indoor positioning and tracking technology. The Bluetooth microchip 112 acts as a Bluetooth beacon and the software application 206 converts the Bluetooth signals received from the microchip 112 into real-world location coordinates for tracking and locating the pacifier 100.

The software application 206 uses Received Signal Strength Indicator (RSSI) value of the received BLE signals 202 to determine if the pacifier 100 is far or near to the electronic device 204. RSSI represents the power of the signal from the Bluetooth microchip 112. The value of RSSI is low when the distance between the electronic device 204 and the pacifier 100 is long, for example greater than 10 meters. The RSSI value is high when the distance between the electronic device 204 and the pacifier 100 is short, for example less than 10 meters.

The other advantages of deploying the Bluetooth Low Energy signal in the present invention is that it consumes low energy and an optional coin cell battery integrated in the base 104 of the pacifier 100 can be used for the Bluetooth microchip 112. The cost of using Bluetooth is less, and the range is enough for indoor tracking of the pacifier 100. In case the pacifier 100 goes out of the Bluetooth range of the electronic device 204, the smartphone application 206 displays the last recorded or past-time location of the pacifier 100 to assist the users in locating the pacifier 100.

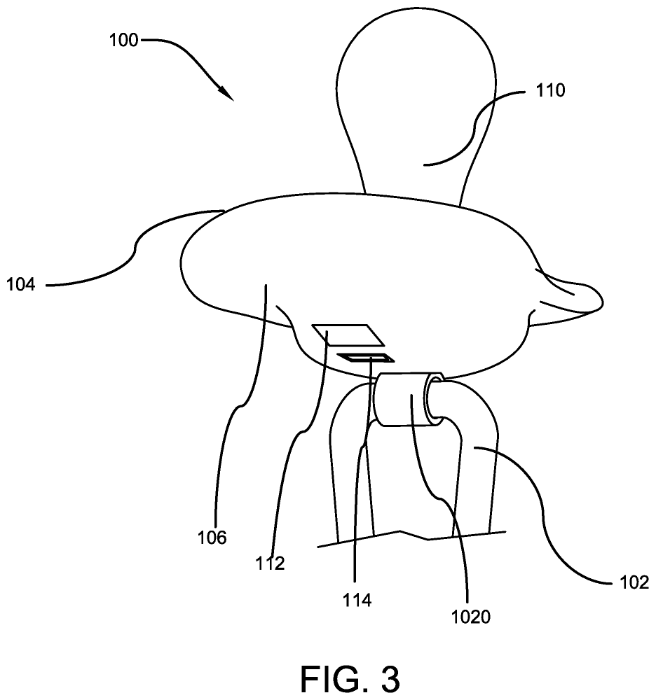

FIG. 3 illustrates a close view of the Bluetooth microchip and micro-USB charging port of the Bluetooth pacifier of the present invention in accordance with the disclosed architecture. The Bluetooth pacifier 100 includes a plastic base 104 having an integrated Bluetooth microchip 112 and micro-USB charging port 114. The Bluetooth microchip 112 allows the pacifier 100 to connect to a smartphone application installed in one or more electronic devices. The Bluetooth microchip 112 transmits real-time, low-energy Bluetooth signals to one or more paired electronic devices in order to allow parents and other users to locate the pacifier 100 with ease.

The micro-USB charging port 114 is configured and designed to connect to a USB charger to charge the Bluetooth microchip 112 of the pacifier 100. Additionally, the micro-USB charging port 114 can be used to connect the pacifier 100 to a pacifier sterilizer using a micro-USB cable.

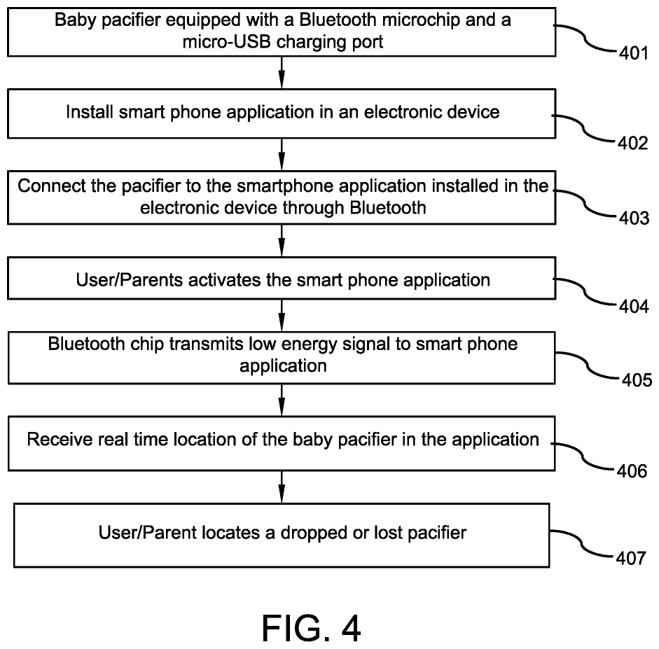

FIG. 4 illustrates a flow diagram showing tracking of the location of the Bluetooth baby pacifier of the present invention using Bluetooth signals in accordance with disclosed architecture. Initially, a Bluetooth pacifier integrated with a Bluetooth microchip and a micro-USB charging port is provided (Block 401). The Bluetooth pacifier is designed to provide real-time location using BLE signals. For receiving real-time alerts on a personal electronic device, a user installs a companion software application in the electronic device (Block 402). Then, the electronic device with installed software application is paired with the Bluetooth pacifier using a wireless communication medium such as Bluetooth Low Energy signals (Block 403). Next, the one or more users activate the smartphone application to track the pacifier (Block 404). The integrated Bluetooth microchip of the pacifier transmits the real-time and periodic low-energy Bluetooth signals to the smartphone application installed in the one or more electronic devices (Block 405). The companion software application installed in the one or more electronic devices receives the BLE signals and processes the signals and the received RSSI values to show the real-time location of the baby pacifier to the user. The real-time location shows the exact location of the baby pacifier (Block 406). Then, the parents and/or other individuals track the real-time location of the pacifier as shown by the software application, and locate the dropped or lost pacifier (Block 407).

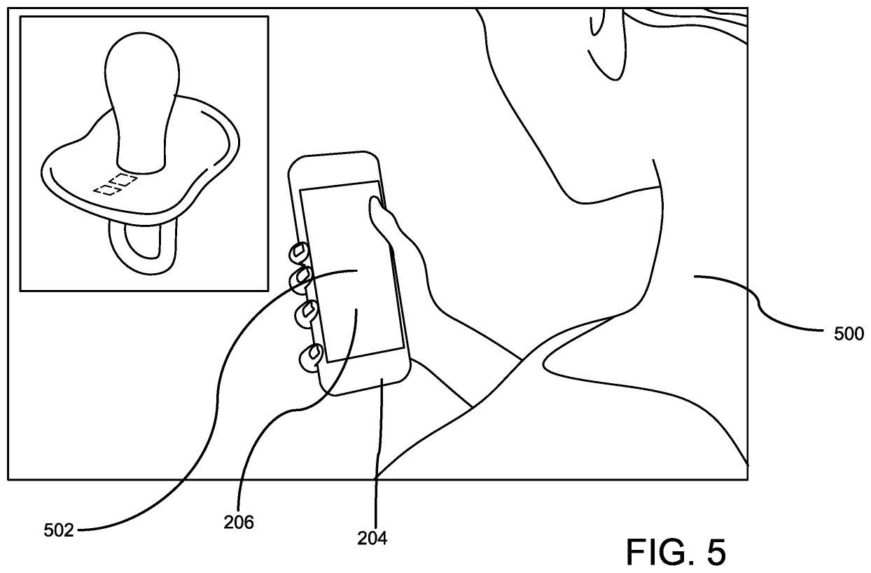

FIG. 5 illustrates a perspective view showing the last recorded location of a connected Bluetooth pacifier on display of an electronic device using the smartphone application in accordance with the disclosed architecture. As described above, the Bluetooth pacifier 100 includes an integrated Bluetooth microchip. The integrated Bluetooth microchip transmits real-time, low-energy Bluetooth signals to a companion smartphone application 206 installed in an electronic device 204 of a user 500 when in BLE range. The BLE range is generally about 30 meters. When the pacifier 100 goes out of the BLE range of the electronic device 204, the smartphone application 206 displays the last recorded location 502 of the pacifier 100 to assist in locating the pacifier 100. The Bluetooth low energy signal provides the last recorded location 502 of the pacifier 100 with accuracy of up to one meter. The user 500 based on the received last recorded location 502 can easily locate the dropped or lost pacifier.

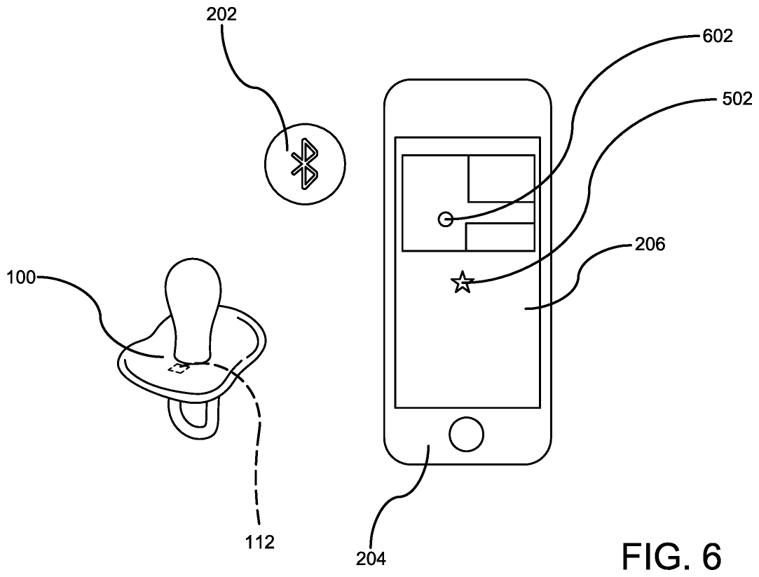

FIG. 6 illustrates a perspective view of the activated integrated Bluetooth chip of the Bluetooth pacifier of the present invention to transmit real-time location in accordance with the disclosed architecture. As shown, the Bluetooth microchip 112 integrated in the base of the pacifier 100 generates a low-energy Bluetooth signal 202. The Bluetooth low-energy signal 202 is a technology for wireless communication that is leveraged to detect and track the location of devices or assets. The real-time location 602 of the pacifier 100 can be displayed along with the last recorded location 502 on the electronic device 204 using the companion software application 206.

A user of the remote electronic device 204 can deactivate operations of the Bluetooth microchip remotely using the companion software application 206. Additional probes or thermometers can be used to measure the temperature of the infant using the pacifier 100 and can be integrated in the base/collar of the pacifier 100, whereby the Bluetooth microchip 112 transmits the temperature of the infant to the software application of one or more paired devices. In one potential embodiment, the Bluetooth microchip 112 acts as a beacon and transmits the BLE signals which are received by the Bluetooth transceiver of the remote electronic device. The software application may generate an alert when the electronic device 204 goes beyond the Bluetooth range of the pacifier 100 to indicate to the user.

It should be noted that the Bluetooth pacifier of all embodiments of the present invention may come as a single unit or come in modular parts with a replaceable nipple or handle. The pacifier may come in a plurality of colors and sizes to meet requirements of children of different ages. The pacifier of the present invention allows the infant to breathe easily without removing the pacifier, and withstands the pressure of chewing and excessive sucking by the infant.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “Bluetooth baby pacifier”, “Bluetooth pacifier”, “pacifier” and “Bluetooth based pacifier” are interchangeable and refer to the Bluetooth pacifier 100 of the present invention.

Notwithstanding the forgoing, the Bluetooth pacifier 100 of the present invention can be of any suitable size and configuration as is known in the art without affecting the overall concept of the invention, provided that it accomplishes the above-stated objectives. One of ordinary skill in the art will appreciate that the size, configuration and material of the Bluetooth pacifier 100 as shown in the FIGS. are for illustrative purposes only, and that many other sizes and shapes of the Bluetooth pacifier 100 are well within the scope of the present disclosure. Although the dimensions of the Bluetooth pacifier 100 are important design parameters for user convenience, the Bluetooth pacifier 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What includes been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A pacifier comprising:
a base;
a chin guard integrally formed with said base;
a nipple selectively connected to said base;
a Bluetooth microchip integrated into said base;
said microchip transmits Bluetooth low energy (BLE) signals to a paired electronic device;
said paired electronic device tracking said microchip;
said tracking includes real-time location of said microchip when said microchip and said paired electronic device are within a first distance of each other;
said tracking includes a past-time location of said microchip when said microchip and said paired electronic device are beyond said first distance of each other; and
wherein said paired electronic device is a smartphone.

2. The pacifier of claim 1, wherein said handle is magnetic for retention of said pacifier to a metal surface.

3. The pacifier of claim 1 further comprising a thermometer integrated into said nipple and said base for monitoring a temperature of a user of said pacifier.

4. The pacifier of claim 3, wherein said microchip transmits Bluetooth low energy signals of said temperature to said paired electronic device.

5. The pacifier of claim 4, wherein said nipple is removable and replaceable.

6. The pacifier of claim 5 further comprising a charging port integrated into said base for charging of said Bluetooth microchip.

7. The pacifier of claim 6, wherein said BLE signals include a Received Signal Strength Indicator (RSSI) value to approximate a second distance between said microchip and said paired electronic device.

8. The pacifier of claim 7, wherein said RSSI value is high when said microchip and said paired electronic device are in close proximity of each other and said close proximity is less than 10 meters.

9. The pacifier of claim 8, wherein said RSSI value is low when said microchip and said paired electronic device are beyond said close proximity of each other.

10. The pacifier of claim 7, wherein said paired electronic device provides an alert when said microchip and said paired electronic device are beyond said first distance of each other.

11. A pacifier comprising:
a base;
a chin guard integrally formed with said base;
a nipple selectively connected to said base;
a Bluetooth microchip integrated into said base;
said microchip transmits Bluetooth low energy (BLE) signals to a paired electronic device;
said paired electronic device tracking said microchip;
said tracking includes real-time location of said microchip when said microchip and said paired electronic device are within a first distance of each other;
said tracking includes a past-time location of said microchip when said microchip and said paired electronic device are beyond said first distance of each other;
wherein said BLE signals include a Received Signal Strength Indicator (RSSI) value to approximate a second distance between said microchip and said paired electronic device;
wherein said RSSI value is high when said microchip and said paired electronic device are in close proximity of each other and said close proximity is less than 10 meters; and
a charging port integrated into said base for charging of said Bluetooth microchip.

12. The pacifier of claim 11, wherein said handle is magnetic for retention of said pacifier to a metal surface.

13. The pacifier of claim 11 further comprising a thermometer integrated into said nipple and said base for monitoring a temperature of a user of said pacifier.

14. The pacifier of claim 13, wherein said microchip transmits Bluetooth low energy signals of said temperature to said paired electronic device.

15. The pacifier of claim 14, wherein said nipple is removable.

16. The pacifier of claim 15, wherein said first distance is 30 meters.

17. The pacifier of claim 16, wherein said paired electronic device is a smartphone

18. The pacifier of claim 17, wherein said RSSI value is low when said microchip and said paired electronic device are beyond said close proximity of each other.

19. The pacifier of claim 18, wherein said paired electronic device provides an alert when said microchip and said paired electronic device are beyond said first distance of each other.

20. The pacifier of claim 14, wherein said nipple is replaceable.
